(19) United States
(12) Patent Application Publication
(10) Pub. No.: US 2010/0137881 A1
(43) Pub. Date: Jun. 3, 2010

(54) Arrangement for Planning and Carrying Out a Surgical Procedure

(76) Inventor: Lukas Kamer, Arth (CH)

Correspondence Address: THE WEBB LAW FIRM, P.C., 700 KOPPERS BUILDING, 436 SEVENTH AVENUE, PITTSBURGH, PA, 15219, US

(21) Appl. No.: 12/527,923

(22) PCT Filed: Feb. 19, 2008
(86) PCT No.: PCT/CH08/00071

§ 371 Date: Aug. 20, 2009

(30) Foreign Application Priority Data
Feb. 22, 2007 (CH) 00302/07

(51) Int. Cl. A61B 19/00; G06T 15/00; G06G 7/60

(52) U.S. Cl. 606/130; 345/419; 703/11

(57) ABSTRACT

The arrangement for planning and carrying out a surgical procedure comprises a medical imaging device, a computer for storing and generating a computer model by means of the image data generated by the imaging device, and a work means for carrying out a processing operation. The work means is configured as a three-dimensional computer mouse and has a connecting means by means of which a part, such as an instrument, a tool, or an implant, can be positioned. The part is illustrated as a template in a computer model of the part of the patient to be treated.

The invention relates to an arrangement for planning and carrying out a surgical procedure, comprising a medical imaging apparatus and a computer for storing and generating a computer model using the image data obtained from the imaging apparatus.

The invention further relates to a method for planning a surgical procedure using such an arrangement, and to a work means for planning and carrying out a surgical procedure.

When planning a surgical procedure, the individual circumstances must be examined with care so as to be able to perform the intended procedure at the suitable site. Such a procedure is, for example, the drilling of a hole in a bone in order to receive an implant for securing a tooth. Account must be taken, for example, of the anatomical relationships, such as the position of the roots of adjacent teeth, the position of the maxillary sinus and of the endosseous blood vessels and nerves, the bone quality and quantity, and also esthetic and functional aspects. Such planning can be very time-consuming and expensive.

The object of the invention is to make available an arrangement and a method of said types that simplify the planning of surgical procedures in the medical sector and particularly the dental sector.

In an arrangement of the type in question, the object of the invention is achieved according to claim 1. In the process planning, the work means has the function of a computer mouse (planning of the procedure on a 3D computer model), and, in the process execution, it has the function of a medical navigation device known to a person skilled in the art (e.g. for the positioning of an implant). The work means thus has both functions at the same time. Moreover, the process planning and process execution can take place bidirectionally, interactively, simultaneously and with referencing.

With the arrangement according to the invention, process steps can be planned on the computer model. When planning has been completed, these steps can be transferred to real life by virtue of the computer being connected to the work means. For example, a hole can be drilled in a bone, or a drill jig can be fitted into a holder. It is also important that working steps can be executed bidirectionally in process planning and process execution, i.e. from the virtual computer model to real life and vice versa. For example, it is thus possible to analyze process steps initially in real life, e.g. position analysis on the patient or on the drill jig by means of the 3D computer mouse, in order then to transfer this information with the computer mouse to the computer model for process planning (from real life to the virtual computer model). Conversely, it is also possible for the process planning carried out on the computer model to be transferred by means of the 3D computer mouse back to the patient or to a drill jig (from the virtual computer model to real life). By real-time coordination of the virtual position with the real position, interactivity is ensured. Simultaneity means that the process planning and process execution can also be carried out at the same time. The process planning and also the process execution take place on the basis of referencing. With the arrangement according to the invention, it is therefore possible to carry out a surgical procedure interactively, simultaneously, bidirectionally and with referencing. Several different referencing means are available, such that substantial adaptation to the nature of the medical or dental procedure is possible.

With the arrangement according to the invention, medical procedures can be made much simpler, in particular can take less time, and, in addition, the surgical trauma can be reduced. This results in a considerable reduction in cost and a simplification of the process compared in particular to known navigation systems and computerized planning systems. The reason for this is in particular that the same apparatus can be used for the process planning and the process execution, and in particular this is based on the concept that the apparatus or work means used can be an optionally modified 3D computer mouse with six degrees of freedom, which is known to a person skilled in the art. Compared to other possible technical solutions, this special work means has the advantage that it has at one and the same time the function of a conventional computer mouse and the function of a medical navigation tool and can at the same time be procured at much less cost than the specially configured work means known to a person skilled in the art, such as navigation tools and other computerized planning systems for the medical sector.

With the arrangement according to the invention, it is possible, for example, that an implant, for example a tooth implant, is planned in the virtual computer model and its position is transferred to real life by a drill that is secured on the work means. Changes by the drill, in particular the drilled hole, can be presented on the computer model. Instead of a drill, however, it is also possible, for example, for a drill sleeve to be transferred to a drill jig. The procurement of much more expensive equipment or of technical aids from external sources is no longer necessary, with the result that the costs and in particular also the time spent can be reduced.

The arrangement can also be used to plan a direct use on the patient. For example, this can be an ENT procedure or an orthodontic procedure. Various instruments and tools, for example drills, positioning aids, pointing instruments and also implants can be secured on the connecting means. The implants can be prosthetic parts or also body parts, for example, bone segments.

According to one development of the invention, the three-dimensionally movable arm is articulated. In particular, the movable arm is composed of several parts that are connected to one another via joints. For position determination, one or more goniometers are provided that determine the angle between two parts of the arm mechanically or electronically. The corresponding values are transferred to the computer. This permits the production of a comparatively inexpensive but very precise work means.

One development of the invention involves a correction process for the arrangement and the method, since potentially relevant positioning inaccuracies may arise between real and virtual positions of the relevant part of the work means and of the positioning instrument, tool or implant. Possible deviations in position can be recorrected since, after the positioning of the instrument, tool or implant, imaging is once again performed in order to evaluate the actual from the target position of the relevant part of the work means and/or of the instrument, tool or implant. This recorrection is done by manual or motorized readjustment of the work means and of the instruments, tools or implants secured thereon, with visualization on a computer model of the actual to the target position.

According to one development of the invention, a fixing element is also arranged on the imaging apparatus or in the proximity thereof (e.g. a wall located close by). Consequently, the imaging apparatus itself and thus the imaging can be referenced, by the patient being temporarily immobilized with referencing means secured on him for the imaging. Such immobilization during the imaging has additionally the advantage that the movement artefacts known to a person skilled in the art are minimized and/or at the same time the medical procedure can be carried out in immediate proximity to the imaging apparatus with referencing.

According to one development of the invention, the arm has a connecting means via which the tool, instrument or implant can be secured in a predetermined position on the arm. These securing means are preferably located at a free end of the arm. The instrument, tool or implant are presented in the computer model as a virtual template. The position of the instrument, tool or implant is synchronized correctly with the position of the virtual template. The position of the template in the computer model in relation to the referencing means thus always corresponds actually to the position of the instrument, tool or implant.

According to one development of the invention, the arm can be fixed in selectable positions. For example, the arm can be fixed for drilling in the intended position or orientation.

According to one development of the invention, the arm can be guided at a front part.

According to one development of the invention, a repositionable holder is provided that is connected to the referencing means. The repositionable holder is, for example, a part that can be secured in a predetermined position on the body of the patient, for example on the jaw. For example, the holder can be a plastic curable compound. The holder is placed, for example, onto the jaw or onto teeth of the jaw, and the plastic compound is deformed. When the compound has hardened, the holder can be removed and can then be secured at any time on the jaw, the referencing means always taking up the same position with respect to the body part or the jaw.

According to one development of the invention, the work means is mounted on a support or plate. A fixing element, on which the referencing means can be positioned, is preferably also arranged on the support. This permits particularly simple and reliable referencing, for example of a holder. In particular, a repositionable holder on the support can be processed in this way by the work means. For example, the holder can be drilled with a hole and provided with a drill sleeve.

In the method according to the invention, according to one development, the position of the work means and of the referencing means is transferred to the computer model and presented in the latter, a template is also presented in the computer model, which template corresponds to a part mountable on the work means, for example an instrument, tool or implant, and the surgical procedure is planned on the basis of the computer model. Process steps performed on the computer can be transferred to real life interactively, simultaneously and with referencing. Medical procedures can be performed from the computer model. These procedures can also be analyzed and, if appropriate, corrected. Values, for example angles, length dimensions and spatial coordinates, can be recorded with the work means, and, furthermore, work can be carried out directly or indirectly with the same work means. Process planning and process execution can thus be coupled via the work means and via the computer. This permits interactive, simultaneous and bidirectional working.

Other advantageous features will become clear from the following description, from the dependent claims and from the drawing.

Illustrative embodiments of the invention are explained in more detail below with reference to the drawing, in which:

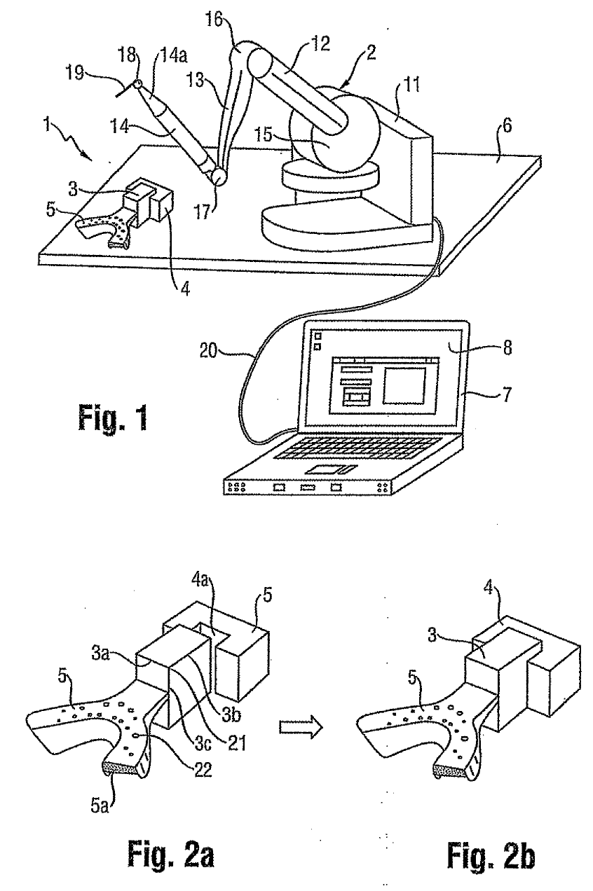

FIG. 1 shows a schematic three-dimensional view of an arrangement according to the invention,

FIG. 2a shows a schematic view of part of the arrangement according to FIG. 1 and illustrates the positioning of a referencing means on a fixing element,

FIG. 2b shows a view according to FIG. 2a, the referencing means being oriented in a predetermined position on the fixing element,

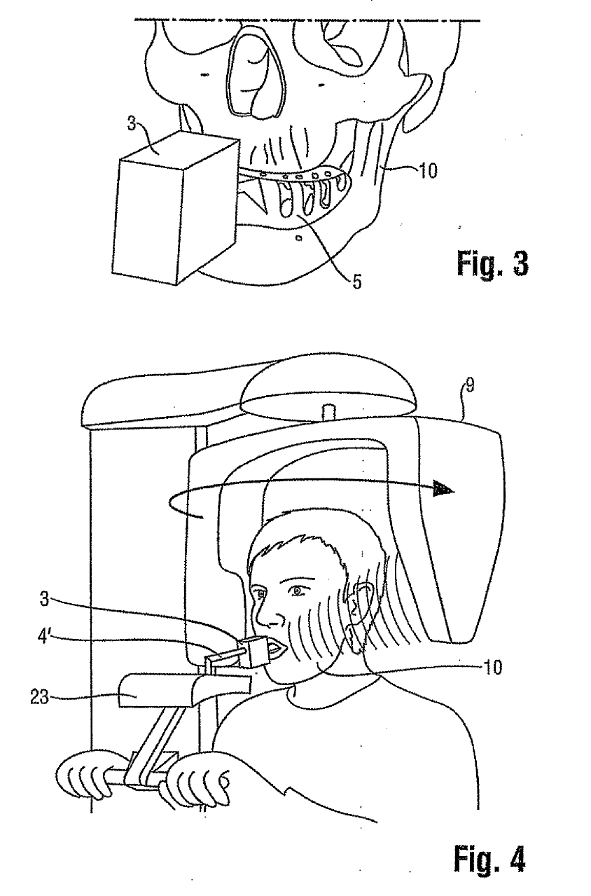

FIG. 3 shows a schematic view of a holder positioned on a jaw, and a referencing means connected to the holder,

FIG. 4 shows a schematic view of an imaging apparatus, with a referencing means secured thereon, and

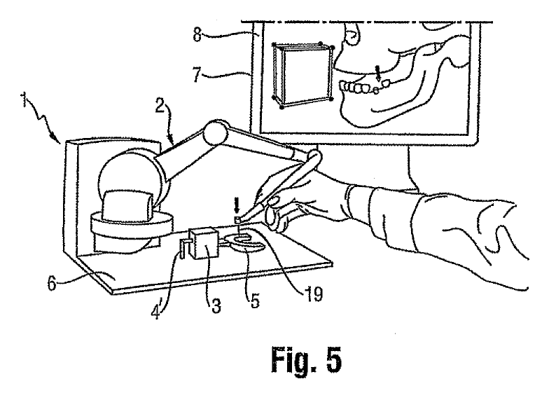

FIG. 5 shows another view of an arrangement according to the invention.

The arrangement 1 according to the invention shown in FIG. 1 comprises a work means 2 which is mounted on a support 6, for example a plate or the like, and which is connected via a signal line 20 to a computer 7, for example a laptop. The work means 2 is additionally connected by cable (not shown here) to a power source for current supply. In principle, however, the work means 2 can also be powered by battery. The work means 2 is controlled by the computer 7, which contains the relevant programs.

The work means 2 comprises a frame 11 on which an arm 12 is mounted. This arm 12 is pivotable about a first joint 15. The movement is preferably effected by hand. In principle, however, a movement by motors is also possible. Moreover, a goniometer, which measures the angle position of the arm 12, is arranged in the joint 15. The corresponding measured values or angles are fed via the signal line 20 to the computer 7. The changes of angle are measured continuously by said goniometer and corresponding measured values transmitted to the computer 7. The joint 15 is a pivot joint, for example. However, it can also be another joint, for example a ball joint or pivot joint. The important thing is that movements are possible in all directions. The work means 2 forms what is called a three-dimensional computer mouse. Such a computer mouse is sold, for example, at www.sensable.com/products or at www.immersion.com/digitizer/microscribe. A computer mouse of this kind, however, does not necessarily have to be produced mechanically with joints.

The arm 12 is preferably designed in several parts and is provided with several joints and arm parts. The illustrative embodiment shown in FIG. 1 comprises a further joint 16, which connects a middle part 13 to the arm 12. The middle part 13 is connected by another joint 17 to a front part 14. The joints 16 and 17 can be connected to a motor and to a goniometer. These motors and goniometers are also connected to the computer 7 for signal transmission. Manual actuation without motors is also conceivable. The motors of the joints 16 and 17 can therefore also be controlled via the computer 7. The angle settings of the joints 16 and 17 are measured, and the measured values are fed to the computer 7. The joints 16 and 17 are pivot joints, for example, but can also be other joints, for example ball joints. The joints 15, 16 and 17 are designed in such a way that a front end 14a of the front part 14 is movable in all directions. On the basis of the goniometer measurements, the corresponding spatial position of the front end 14a can be calculated in the computer 7. If the end 14a is moved by hand from a first position A to a second position B, the corresponding changes in position are transferred continuously and in real time to the computer. In addition to said joints, other joints are also possible. For example, a pivot joint with a vertical axis is conceivable about which the arm 12 can swivel.

Arranged at the end 14a of the front part 14 there is a securing means 18 via which a part 19 can be connected to the arm 12. The part 19 is in particular an instrument, a tool or an implant. For example, the instrument can be a pointing instrument, a probe or the like. The tool is, for example, a drill or a reamer. The implant can be any medical implant, for example a dental implant. However, the part 19 can also be an aid, for example a drill sleeve. The important thing is that the part 19 can be secured in a predetermined position and orientation at the end 14a. By means of said goniometers, the computer 7 can calculate the position and orientation of the part 19 and can also control this position and orientation. The position and orientation of the part 19 can also be displayed on a screen 8 of the computer 7. If the position of the part is changed, this change is visible substantially simultaneously on the screen 8. In addition to the part 19, other parts of the work means 2 can also be displayed on the screen 8. These displays can also be schematic. The securing means 18 can be formed, for example, as a clip or as a locking device or the like. With the securing means 18, the part can be secured releasably in a predetermined position on the front part 14.

Arranged on the support 6 there is a fixing element 4 on which a referencing means 3 can be positioned. The positioning of the referencing means 3 on the fixing element 4 is shown schematically in FIGS. 2a and 2b. The fixing element 4 for this purpose has a recess 4a, which is at least in part designed corresponding to the referencing means 3. The fixing element 4 is preferably connected fixedly to the support 6 and is at a defined distance from the work means 2. By contrast, the referencing means 3 is movable and is mounted on a holder 5. The fixing element 4 is only depicted schematically here. The connection between it and the referencing means 3 can be of any desired type. For example, this connection can be a plug-in connection, a locking connection or, for example, also a bayonet connection or the like. The important thing is that the position of the referencing means 3 can be fixed exactly by the fixing element 4 relative to the work means 2. The referencing is done here, for example, with respect to the edges 3a, 3b and 3c, which are shown in FIG. 2a and which have a common corner 21 and are preferably at right angles to one another. The support 6 thus connects the referencing means 3 fixedly with respect to the work means 2. FIGS. 4 and 5 show an alternative fixing element 4, which has an angled configuration but otherwise has the same function as the fixing element 4.

The holder 5 serves, for example, to receive a drill sleeve 22. This drill sleeve 22 is used to guide a drill with which a hole for receiving an implant is drilled at a predetermined site in a jaw bone. Such drill sleeves 22 and implants are well known in dentistry and do not therefore need to be explained in further detail here.

The holder 5 is designed such that it can be secured on a jaw 10 in a repositionable manner, as is shown schematically in FIG. 3. For this purpose, the holder 5 has a plastic and curable compound 5a, for example on the underside. Accordingly, when the holder 5 is placed on the jaw 10, this compound 5a is plastically deformed, in particular by the teeth of the jaw 10. When the holder 5 is removed from the jaw 10, the plastic deformation is maintained and it can be arranged in the same position as before on the jaw 10. Since the referencing means 3 is connected securely to the holder 5, the referencing means 3 can therefore also be secured in a repositionable manner on the jaw 10.

The arrangement according to the invention also comprises an imaging apparatus 9 shown schematically in FIG. 4. This is, for example, a computed tomograph, a cone beam computed tomograph, a magnetic resonance imaging apparatus or an ultrasound apparatus. A three-dimensional image can be recorded by this imaging apparatus 9 and stored in the computer 7. FIG. 4 shows a schematic view of an arrangement with which an image of a jaw 10 can be produced, with a holder 5 repositioned on the jaw 10, and of a referencing means 3. The referencing means 3 is positioned on a fixing element 4′ which is connected fixedly to the imaging apparatus 9 via a holder 23. With the fixing element 4′ and the holder, the patient can be immobilized during the imaging, as a result of which artefacts that are known per se can be substantially reduced. It would also be conceivable for the fixing to be effected not on the imaging apparatus, but instead on a structural wall, for example. In this image, the abovementioned three edges 3a, 3b and 3c and the corner 21 can be recorded as coordinates. The referencing means 3 can therefore be secured, according to FIG. 1, on a support 6 or, according to FIG. 4, directly on the body to be treated or on a jaw 10. The processing with the work means 2 takes place in one case outside the body, and in the other case directly on the body.

The arrangement 1 according to the invention makes it possible to control the work means 2 by the computer 7 and also allows process steps that have been worked out on a computer model to be transferred back to real life interactively, simultaneously and with referencing. In this transfer, the drill sleeve 22, for example, is secured on the holder 5, or a surgical procedure is carried out, for example an implant is inserted or, as has been explained above, a hole for receiving an implant is drilled in the jaw 10. The execution of the processes can be bidirectional, i.e. from the virtual computer model to real life, and vice versa. In order to control the part 19, the angles, length dimensions and spatial coordinates determined by the goniometers are recorded by the work means 2 and transferred to the patient either directly or indirectly, for example by means of the holder 5. Process planning and process execution are coupled by the work means 2 to the computer model. Process steps, for example the drilling of the jaw 10, can thus be carried out interactively, simultaneously, bidirectionally and under referenced conditions.

The arrangement 1 can be used, for example, to plan and carry out the fitting of an implant for securing an artificial tooth. The planning is done on the basis of a computer model on the computer 7. The computer model is based on a three-dimensional image that was generated by the imaging apparatus 9. Using this computer model, the suitable position of the intended artificial tooth can be planned with precision on the screen 8. The hole provided for this purpose can be drilled directly on the jaw 10 or via the holder 5. In the computer model, the part 19 is presented as a template. The real drill tip and the drill axis then correspond exactly to the drill tip and the drill axis in a virtual model. In carrying out the work step, the corresponding work process can be monitored and, if appropriate, also corrected, controlled and optimized. Instead of the transfer from the virtual computer model to real life, the bidirectionality means that the reverse is also possible. Thus, a work process can first be carried out and this controlled and optimized in a process planning on the computer model. Moreover, by virtue of the simultaneity, process planning and process execution can also be done at the same time. The arrangement 1 thus makes it possible for process planning and process execution to be transferred interactively, simultaneously, bidirectionally and with referencing from real life to a virtual computer model and vice versa. An important advantage of this is that the choice of different referencing variants is possible. In particular, an adapted approach is possible depending on the nature of the medical or dental procedure.

FIG. 5 shows once again the interactive planning and carrying out of the method according to the invention using the work means 2 configured as a three-dimensional computer mouse. The position of the implant and the axis is transferred from the computer model to the repositionable holder 5 by drilling and fitting of a drill sleeve 22. The planning and transfer is preferably assisted by a haptic position guide (haptic positional sensing) of the computer mouse. After the planning, the work means can be used as a surgical guide means during an operation.

### LIST OF REFERENCE SIGNS

1 arrangement

2 work means

3 referencing means

3a edge

3b edge

3c edge

4 fixing element

4a recess

5 holder

5a compound

6 support

7 computer

8 screen

9 imaging apparatus

10 jaw

11 frame

12 arm

13 middle part

14 front part

14a end

15 joint

16 joint

17 joint

18 securing means

19 part

20 line

21 corner

22 drill sleeve

23 holder

1-22. (canceled)

23. An arrangement for planning and carrying out a surgical procedure, comprising a medical imaging apparatus; a computer for storing and generating a computer model using the image data obtained from the imaging apparatus; and a work means for carrying out a processing operation, wherein said work means is configured as a three-dimensional computer mouse and has connecting means for securing a part that is an instrument, tool or implant.

24. The arrangement as claimed in claim 23, wherein said work means comprises an articulated arm.

25. The arrangement as claimed in claim 23, wherein said part is secured in a predetermined position and orientation on a front end of the work means, and in said part is presented in the computer model as a virtual template.

26. The arrangement as claimed in claim 25, wherein the three-dimensional position of the part is synchronized with the position of the virtual template in the computer model.

27. The arrangement as claimed in claim 24, wherein said arm is fixed in selectable positions.

28. The arrangement as claimed in claim 24, wherein said arm comprises at least one joint and, for position determination, at least one goniometer.

29. The arrangement as claimed in claim 24, wherein said arm can be guided by hand at a front part.

30. The arrangement as claimed in claim 23, wherein a referencing means is provided on which the work means can be referenced.

31. The arrangement as claimed in claim 23, wherein a repositionable holder is provided that can be repositioned on a body part of a patient.

32. The arrangement as claimed in claim 31, wherein said repositionable holder can be repositioned on a jaw.

33. The arrangement as claimed in claim 32, wherein said repositionable holder is designed as a drill jig.

34. The arrangement as claimed in claim 23, wherein said work means is mounted on a support.

35. The arrangement as claimed in claim 34, wherein a fixing element, on which the referencing means can be positioned, is arranged on the support.

36. A method for planning a surgical procedure said arrangement comprising:
providing at least one medical imaging apparatus and a computer;
generating a computer model using image data obtained from an imaging apparatus;
storing said computer model; and
providing a work means configured as a three-dimensional computer mouse, comprising a part for the surgical procedure, wherein said part comprising an instrument, a tool or an implant secured on the work means.

37. The method as claimed in claim 36, presenting spatially with a template in a computer model generated with the aid of the imaging apparatus the intended real position of the part mountable on the work means.

38. The method as claimed in claim 36, wherein said real position is a position on a body part of a patient.

39. The method as claimed in claim 36, wherein said real position is a position on a repositionable holder.

40. The method as claimed in claim 36, wherein said real position is a position of a drill sleeve or of a drill.

41. The method as claimed in claim 36, further comprising immobilizing the patient's head during imaging by fixing on the imaging apparatus or on a wall.

42. A work means for planning and carrying out a surgical procedure, wherein it is configured as a three-dimensional computer mouse, and in that it has connecting means for securing a part with which the surgical procedure can be carried out.

43. The work means as claimed in claim 42, wherein it has an articulated arm.

44. The work means as claimed in claim 42, wherein the connecting means is arranged on a free end at the front.
